(19) United States
(12) Patent Application Publication
(10) Pub. No.: US 2017/0088846 A1
(43) Pub. Date: Mar. 30, 2017

(54) Functional Analysis of LAZY1 in Arabidopsis thaliana and Prunus Trees

(71) Applicant: The United States of America, as Represented by the Secretary of Agriculture, Washington, DC (US)

(72) Inventors: Christopher D. Dardick, Shenandoah Junction, WV (US); Ralph Scorza, Shepherdstown, WV (US); Courtney A. Hollender, Brunswick, MD (US)

(21) Appl. No.: 14/866,828

(22) Filed: Sep. 25, 2015

(51) Int. Cl. C12N 15/82

(52) U.S. Cl. CPC C12N 15/8218; C12N 15/8222

(57) ABSTRACT

Technology to optimize plant architecture is critical for future efforts to increase planting density in a wide range of crops. Little is known regarding the molecular mechanisms governing this basic plant developmental feature, particularly in fruit trees. Recently, a pair of distantly related genes called LAZY1 and TILLER ANGLE CONTROL 1 (TAC1) was shown to have opposing effects on lateral branch angle in monocots and dicots. We have characterized the LAZY1 gene in both Arabidopsis and plum (Prunus domestica) and assessed its functional relationship with TAC1. Both lazy1 and tac1:lazy1 Arabidopsis plants showed a previously unreported weeping phenotype. Transgenic plum lines silenced for LAZY1 showed horizontal branch angles, sometimes marked by rootward lateral branch growth. Our results establish that manipulation of LAZY1 gene function results in changes in tree shape and can be used to engineer fruit or ornamental trees with desired branch angles.

### BACKGROUND OF THE INVENTION

Field of the Invention

This invention relates to the gene LAZY1 and its role in controlling the orientation of lateral branch growth in plants and to new methods of changing the architecture of fruit and ornamental trees by silencing LAZY1.

Description of the Relevant Art

Changes to plant architectural features can dramatically improve crop productivity. Plant size and shape, collectively referred to as architecture, have a profound impact on agricultural productivity. It is a limiting factor for planting density and crop yield. The green revolution of the mid-1900s was led, in part, by the creation of semi-dwarf cereal varieties with thicker and sturdier stalks that could support more grain (Allard, R. W. 1961. Crop Sci. 1:127-133; Dalrymple, D. G. 1980. USDA, AER No. 425, Office of International Cooperation/Agency for International Development. Washington, D.C., June, 155 pp.; Schertz et al. 1974. Crop Sci. 14:106-109). Since the 1930s, maize planting density has more than doubled (Duvick, D. N. 2005. Adv. Agron. 86:83-145). One factor for this increase was the development of varieties with upright leaf angles that allow efficient light capture under crowded conditions (Pendleton et al. 1968. Agron. J. 60:422-424). Building on these successes, further improvements to plant architecture are being sought for important crops such as rice in attempt to keep pace with the growing demand for food (Cheung, F. 2014. Nature 514 (7524):S60-1; Eratum in Nature 515 (7528):492). In the face of climate change, erosion of topsoil, need for reduced chemical inputs, and scarcity of arable land, the United Nations Food and Agriculture Organization (FAO) has projected that food production must increase by 70% to feed the world's population in 2050 (Conforti, P. 2011. Report of the Food and Agriculture Organization of the United Nations. Rome, Italy).

While substantial architectural improvements have been made in staple crops such as corn and rice, many crops still remain relatively unchanged from their wild progenitors. This is true for most dicots, particularly fruit and nut crops which are difficult and slow to breed. In place of genetic improvements, these agricultural production systems have adapted by using costly horticultural manipulations including training, trellising, grafting onto dwarfing rootstocks, and pruning. These efforts are labor intensive and require excessive use of fuel, fertilizer, chemical inputs, labor, and land. Newer complex horticultural systems that manipulate tree size and shape to enable high density planting are beginning to take hold such as the apple spindle production system (Robinson et al. 2006. New York Fruit Qrtrly 14:21-28).

The orientations of lateral organs are influenced by a number of factors including nutrition, light, temperature, mechanical stress, and crowding (Tomlinson, P. B. 1983. American Scientist 71:141-149). This built-in plasticity allows plants to optimize their growth in response to environmental conditions. Architectural changes can be brought about in two ways. The first is a change in tissue rigidity through alterations in turgor pressure or tensile strength. For example, nastic movements of leaves are caused by changes in turgor pressure at leaf petioles, promoting differential cell expansion that can push leaves up or down (Koller, D. 1990. Plant, Cell & Environment 13(7):615-632). Additionally, the production of reaction wood (primarily in trees) provides increased tensile strength to pull or push heavy branches up in response to the mechanical stress of increased weight (Guerriero et al. 2014. Tree Physiology 34:839-855). The second way includes changes to plant sensory systems that stimulate differential cell expansion and/or division. These movements, called ‘tropisms’, allow plants to sense environmental signals such as light (phototropism) or gravity (gravitropism) and respond accordingly. Shade avoidance is one well-studied example of how plant growth is altered in response to changes in light. Filtering of light through a leaf canopy reduces the red to far red light ratio, which is sensed by light receptors that can trigger a wide-range of developmental adaptations like altered stem and leaf petiole growth (Franklin, K. A. 2008. New Phytologist 179(4):930-944).

Gravitropism has been implicated in setting the orientation of lateral shoots but the underlying mechanisms are complex (Hollender and Dardick. 2015. New Phytologist 206:541-556). There are three main components of lateral shoot orientation: 1) the angle made by the shoot and primary stem 2) the angle between the growth trajectory of the lateral shoot and the main stem, termed the equilibrium angle (Wilson, B. F. 2000. Amer. J. Bot. 87:601-607), the angle of inclination (Brown, C. L. 1971b. In: Trees: Structure and Function. Zimmerman and Brown, Eds. Springer-Verlag, New York, N.Y., pp. 125-167) and the gravitational set point angle (Digby and Firn. 1995. Plant, Cell & Environment 18:1434-1440; Roychoudhry et al. 2013. Curr. Biol. 23:1497-1504) and 3) the geotropic angle of the tip of a branch to the main trajectory. The non-vertical growth behavior of lateral shoots is potentially caused by an unknown anti-gravitational plant growth offset (Roychoudhry et al. 2013, supra).

Nearly all tropism-based plant movements have been attributed, at least in part, to auxin signaling and transport (Friml, J. 2003. Curt. Opin. Plant Biol. 6(1):7-12). Auxin is primarily produced at the plant apex and transported rootward via a set of directionally oriented transporters such as the PIN-FORMED proteins (PINs). In the root tip, auxin is then transported away from the apex and upward in the outer tissues. During various stages of development and in response to stimuli such as light or gravity, PINs reorient to alter the direction of auxin transport (Kfreek et al. 2009. Genome Biol. 10(12):249). The resulting changes in auxin gradients are thought to drive cell differentiation to alter cell growth, expansion, and/or lignification. It is important to note that although auxin has been implicated in these processes, its relationship to other signals such as other hormones, sugars, or nutrients remains unclear (Mason et al. 2014. Proc. Natl. Acad. Sci. USA 11:6092-6097).

What we've learned from successes in maximizing crop productivity is that specific crop architectural features, such as lateral branch orientation can be limiting factors. The number, size, position, and orientation of lateral organs including shoots, leaves, and roots strongly influences planting density, light interception, and soil utilization. While substantial progress has been made in horticulture and breeding, sizeable knowledge gaps regarding plant architecture still hamper these efforts.

### SUMMARY OF THE INVENTION

We have identified the LAZY1 gene (SEQ ID NO:1) of the IGT family and confirmed that silencing its expression results in Prunus trees having horizontally oriented branches and, in some instances, a weeping appearance.

In accordance with this discovery, it is an object of the invention to provide a method to routinely control an architectural aspect of Prunus trees comprising silencing of the expression of the LAZY1 gene (SEQ ID NO:1) in Prunus trees or germplasm to obtain Prunus trees where branches are horizontally oriented and leaves have a rootward orientation while still retaining normal flower and fruit development, thus resulting in the improved ability to train the branches on trellises or wires eliminating the need and cost for continual manipulation and pruning of plants that have been trellised and wired to obtain desired growth direction.

It is another object of the invention to provide an isolated or recombinant polynucleotide molecule comprising a 306 consecutive base pair fragment (SEQ ID NO:4) of the LAZY1 gene (SEQ ID NO:1).

It is an additional object of the invention to provide a hairpin nucleic acid construct comprising a LAZY1 polynucleotide gene sequence comprising a 306 consecutive sense nucleotide fragment (SEQ ID NO:4) of the LAZY1 gene of Prunus and the antisense-complement thereof, such that first and the second polynucleotide sequences hybridize when transcribed into a ribonucleic acid to form the hairpin-like double stranded ribonucleotide molecule.

It is an object of the invention to provide transformed Prunus plant cells and Prunus plants having horizontally oriented branches and leaves having a rootward orientation and retaining normal flower and fruit development.

It is a further object of the invention to manipulate tree shape resulting in horizontal branch growth or a weeping phenotype in Prunus cultivars including Prunus persica (peach), Prunus domestica (plum), Prunus avium (cherry), Prunus salicina (Japanese plum) and Prunus armeniaca (apricot).

It is an additional object of the invention to manipulate the degree of silencing of LAZY1 such that strong silencing may be desired for ornamentals and moderated silencing for orchard applications.

It is another object of the invention to provide a method of producing a Prunus plant having the characteristics of weeping tree architecture comprising: constructing a recombinant vector comprising a construct comprising the 306 base pair consecutive nucleotide fragment (SEQ ID NO:4) of the LAZY1 gene of Prunus, transforming Prunus plant cells with the recombinant vector and expressing in the plant said construct encoding the LAZY1 gene sequence comprising a 306 bp consecutive sense nucleotide fragment of the LAZY1 gene of Prunus and the antisense-complement thereof, wherein the expressing induces RNA interference (RNAi) in the plant resulting in plants having the characteristics of weeping architecture, where the branches have a horizontal orientation.

It is another object of the invention to provide a transgenic Prunus plant, produced by the methods of the invention, or the progeny thereof, comprising: the RNAi construct of the invention, said plants exhibiting changed plant architecture with horizontally oriented branches compared to a wild-type non-transformed Prunus plant.

It is an additional object of the invention to provide a transgenic Prunus cell comprising the RNAi construct of the invention, wherein the transgenic plant regenerated from said cell exhibits suppression of the LAZY1 gene, said RNAi construct comprising the fragment of SEQ ID NO:4 resulting in a plant demonstrating changed plant architecture with horizontally oriented branches and leaves having a rootward orientation, relative to the wild-type Prunus plant.

It is yet another object of the invention to provide plants, plant cells, and plant parts, and plant seeds which have been transformed by the LAZY1 RNAi construct of the invention.

Other objects and advantages of this invention will become readily apparent from the ensuing description.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIGS. 1A, 1B and 1C depict the standard wild type Arabidopsis plant (FIG. 1A) and mutant phenotypes associated with loss of expression of TAC1 (FIG. 1B) or LAZY1 (FIG. 1C) in Arabidopsis plants.

FIGS. 2 Left and 2 Right depict gravitropic bending of Col-0, tac1, lazy1, and lazy1::tac1 mutants. FIG. 2 Left is a graph showing area of curve measurements taken from Col-0, tac1, lazy1, and lazy1::tac1 double mutants over the course of 11 hrs. FIG. 2 Right shows that lazy1 and lazy1::tac1 mutants have delayed bending; however, they reach a vertical orientation by 24 hrs.

FIG. 3 shows that lazy1 plants display a weeping phenotype under high light. Growth of lateral shoots in lazy1 and lazy1::tac1 plants is pendulous under high light.

FIGS. 4A, 4B and 4C depict the characterization of transgenic plum lines silenced for LAZY1. FIG. 4A shows images of architectures of two typical plum lines silenced for LAZY1. FIG. 4B shows LAZY1 expression in silenced lines measured via qPCR. FIG. 4C shows leaf orientation phenotypes observed in LAZY1 silence lines.

FIGS. 5A and 5B show qPCR data showing differential regulation of TAC1 by light and gravity. FIG. 5A depicts dark repression of TAC1 in shoots of Arabidopsis and peach. FIG. 5B depicts Arabidopsis TAC1 repression during time course after dark incubation.

FIG. 6A shows subcellular localization of peach LAZY1 and TAC1 proteins in onion epidermal cells. FIG. 6B depicts pairwise Y2H tests showing self-interaction of peach LAZY1 (all grown on −His selection).

### DETAILED DESCRIPTION OF THE INVENTION

Currently, little is known about the molecular mechanisms governing lateral organ orientation (Roychoudhry and Kepinski. 2015. Curr. Opin. Plant Biol. 23: 124-131). Our recent work has uncovered a novel gene family called IGT, named for a conserved motif GL(A/T)IGT found in domain II of diverse TAC1 proteins, that influences the orientation of roots, shoots, and flowers (Dardick et al. 2013. The Plant J. 75(4):618-630). IGT family members were first identified as monocot-specific genes in rice but were not known to be part of same gene family due to an overall lack of sequence conservation. Currently, the mechanisms of IGT gene function and the extent to which they influence global plant architecture remain poorly defined.

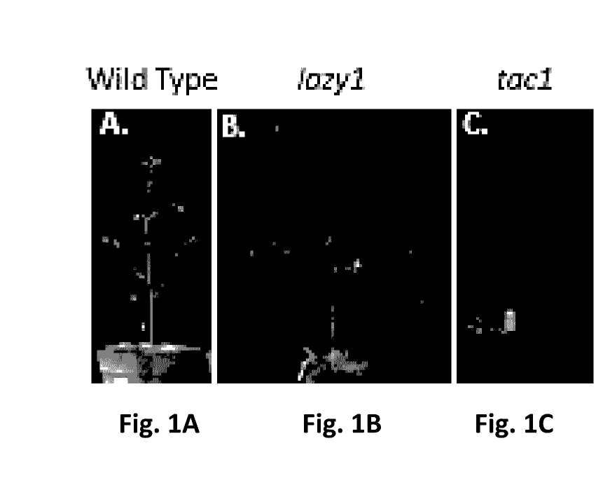

LAZY1, the first reported gene in the IGT family, has been shown to control the orientation of tillers in rice and maize as well as lateral shoots of Arabidopsis. Mutations that eliminate or reduce LAZY1 expression led to horizontally oriented lateral tillers or shoots, an effect associated with a loss of gravitropic responses as well as decreased lateral auxin transport (FIG. 1A) (Li et al. 2007. Cell Res. 17:402-410; Yoshihara and Lino. 2007. Plant Cell Physiol. 48:678-688; Dong et al. 2013. Plant Physiol. 163(3):1306-1322). In maize, loss of LAZY1 was also found to impact inflorescence development (Dong et al. 2013, supra). Reduced or abolished expression of a second IGT gene, TAC1 (TILLER ANGLE CONTROL), was shown to produce the opposite phenotype as LAZY1 in rice, maize, Arabidopsis, and peach (Yu et al. 2007. Plant J. 52(5):891-898; Ku et al. 2011. PLOS One 6(6):e20621; Dardick et a. 2013, supra). TAC1 mutations resulted in upright tiller or lateral branch angles (FIG. 1B) (Yu et al. 2007, supra; Ku et al. 2011, supra; Dardick et al. 2013, supra). The tac1 mutations in rice and maize were found to be largely responsible for the upright architectures that enable high density production of these crops (Yu et al. 2007, supra; Ku et al. 2011, supra). Likewise, peach tac1 mutations are being exploited for high density orchard systems (Scorza et al. 2006. Acta Hort. 713:61-64). Interestingly, the effects of tac1 mutations in peach were not limited to lateral branch angles but also effected the orientation of floral meristems and root structures (Tworkoski and Scorza. 2001. J. Amer. Hort. Sci. 126:145-155; Dardick et al. 2013, supra).

The only known functional domain found within LAZY1 is an EAR motif (ethylene response factor-associated amphiphilic repression) at the C-terminus of the protein (typically LxLxL). EAR motifs often serve as repression domains that function by recruiting a co-repressor such as TOPLESS (TPL) (Szemenyei et al. 2008. Science 319 (5868): 1384-1386). For example, in auxin signaling, TPL and Aux/IAA proteins interact via the EAR domain and together inhibit the activity of Auxin Response Factors (ARFs) which transcriptionally activate auxin signaling (Szemenyei et al. 2008, supra). This mechanism of auxin regulation is at the heart of a wide range of plant developmental processes and is widely conserved, having been shown to operate in primitive plants such as moss (Causier et al. 2012. Plant Signaling & Behavior 7(3):325-328).

Here we evaluated the functions of LAZY1 in both Arabidopsis and plum. In both plant species, loss of LAZY1 resulted in horizontally oriented branches. In contrast to a previous report, gravitropic responses in a lazy1 Arabidopsis knock-out mutant was not abolished but only reduced or delayed. lazy1 Arabidopsis plants were also found to be highly sensitive to light as plants grown under higher light conditions displayed a distinctive weeping phenotype where all shoot meristems grew rootward. A double mutant of both tac1 and lazy1 exhibited the lazy1 mutant phenotype, suggesting lazy1 is epistatic to tac1. Similar to Arabidopsis, the branches of LAZY1-silenced plums grew more horizontally than upward. In addition, LAZY1-silenced plums also had leaves with a more rootward orientation suggesting that LAZY1 function in plum is not limited to the control of branch angles.

The results presented here confirm that LAZY1 function is conserved across dicots and provides further insight into LAZY1 function and its relationship to TAC1. tac1;lazy1 plants were phenotypically identical to lazy1 mutants alone, establishing lazy1 as epistatic to tac1. We previously hypothesized that TAC1 functions as a negative regulator of LAZY1 either directly or indirectly. These data are consistent with that hypothesis and indicate that TAC1 functions upstream of LAZY1. Yeast two-hybrid results indicate that the TAC1 and LAZY1 proteins do not directly interact with each other. However, protein localization studies suggest they reside in the same cellular locations including the nucleus, plasma membrane, and microtubules. This finding implies they likely function together to control branch angle but perhaps interact via a third protein partner or only under specific environmental conditions.

lazy1 Arabidopsis mutant plants displayed a distinctive light-dependent weeping phenotype. This phenotype was likewise observed in tac1;lazy1 double mutants indicating that it is not caused by or does not require TAC1. At this time it is not clear whether the rootward growth of lateral shoots under high light was a consequence of a reversal of normal gravitropism or by negative phototropism, a phenomenon whereby shoots grow away from, instead of towards, light. Carefully controlled phototropism experiments will be necessary to assess the cause of this phenotype; however, it can be concluded that LAZY1 plays an important role in plant growth responses to light. To further evaluate this function of LAZY1, we surveyed LAZY1 and TAC1 gene expression under light and dark conditions via qPCR. LAZY1 did not significantly respond to any of the conditions tested and remained relatively constant in all experiments. In contrast, TAC1 expression was found to be light dependent in both peach and Arabidopsis. TAC1 transcript was significantly reduced after 24 hrs of dark treatment and was all but gone by 48 hrs. This suggests that TAC1 response to light is relatively slow and does not significantly change during normal day-night cycles. Such responses are similar to those involved in shade-avoidance syndrome. Shade avoidance is regulated by a set of light receptors called phytochromes that detect permanent changes in light quality. Collectively, these data suggest that LAZY1 and TAC1 may operate as part of a known or parallel shade avoidance signaling cascade. Such a model could account for how directional growth of lateral shoots is regulated by the environment. Loss of TAC1 expression would predictably promote vertical branch growth via LAZY1. In contrast, TAC1 would suppress LAZY1 under normal light conditions, promoting horizontal growth. This model could likewise explain the long known growth dynamics of lateral shoots of plants and trees which generally grow vertically in the dark or under low light and grow more horizontally under higher light conditions. It is important to point out however that the weeping phenotype displayed by lazy1 and tac1;lazy1 mutants indicates that additional mechanisms operate to control lateral shoot growth that are not dependent on either TAC1 or LAZY1.

The ability to manipulate plant size and shape offers tremendous possibilities to engineer plants suited to high density production systems and/or mechanization. Our finding that plum trees silenced for LAZY1 produce branches that are horizontally oriented may have direct application for managed orchards. Trees having reduced LAZY1 function should be more amenable to training or trellising, having branches that do not reorient growth when moved.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. 1987. Meth. Enzymol. 143:277) and particle-accelerated or “gene gun” transformation technology (Klein et al. 1987. Nature (London) 327:70-73; U.S. Pat. No. 4,945,050, incorporated herein by reference). Additional transformation methods are disclosed below. Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al. 1985. Supp. 1987. Cloning Vectors: A Laboratory Manual; Weissbach and Weissbach. 1989. Methods for Plant Molecular Biology, Academic Press, New York; and Flevin et al. 1990. Plant Molecular Biology Manual, Kluwer Academic Publishers, Boston. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

As used herein, the terms “nucleic acid molecule”, “nucleic acid sequence”, “polynucleotide”, “polynucleotide sequence”, “nucleic acid fragment”, “isolated nucleic acid fragment” are used interchangeably herein. These terms encompass nucleotide sequences and the like.

The term “isolated” polynucleotide refers to a polynucleotide that is substantially free from other nucleic acid sequences, such as other chromosomal and extrachromosomal DNA and RNA, that normally accompany or interact with it as found in its naturally occurring environment. However, isolated polynucleotides may contain polynucleotide sequences which may have originally existed as extrachromosomal DNA but exist as a nucleotide insertion within the isolated polynucleotide. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

As used herein, “recombinant” refers to a nucleic acid molecule which has been obtained by manipulation of genetic material using restriction enzymes, ligases, and similar genetic engineering techniques as described by, for example, Sambrook et al. 1989. Molecular Cloning: A Laboratory Manual, Second Edition, Cold Spring Harbor Laboratory Press, Cold Spring Harbor, N.Y. or DNA Cloning: A Practical Approach, Vol. I and II (Ed. D. N. Glover), IRL Press, Oxford, 1985.

A “construct” or “chimeric gene construct” refers to a nucleic acid sequence encoding a protein, here the LAZY1 protein, operably linked to a promoter and/or other regulatory sequences.

As used herein, the term “express” or “expression” is defined to mean transcription alone. The regulatory elements are operably linked to the coding sequence of the LAZY1 gene such that the regulatory element is capable of controlling expression of LAZY1 gene. “Altered levels” or “altered expression” refers to the production of gene product(s) in transgenic organisms in amounts or proportions that differ from that of normal or non-transformed organisms.

As used herein, the terms “encoding”, “coding”, or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

The term “operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a nucleotide sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. The tissue-specificity of a promoter, for example, is exemplified by the promoter sequence (described above) which specifically induces gene expression in root tips. Promoters that cause a nucleic acid fragment to be expressed in most cell types at most times are commonly referred to as “constitutive promoters”. New promoters of various types useful in plant cells are constantly being discovered; numerous examples may be found in the compilation by Okamuro and Goldberg. 1989. Biochemistry of Plants 15:1-82. It is further recognized that since in most cases the exact boundaries of regulatory sequences have not been completely defined, nucleic acid fragments of different lengths may have identical promoter activity.

“RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from posttranscriptional processing of the primary transcript and is referred to as the mature RNA. “Messenger RNA (mRNA)” refers to the RNA that is without introns and that can be translated into polypeptides by the cell. “cDNA” refers to a DNA that is complementary to and derived from an mRNA template. The cDNA can be single-stranded or converted to double stranded form using, for example, the Klenow fragment of DNA polymerase I. “Sense” RNA refers to an RNA transcript that includes the mRNA and so can be translated into a polypeptide by the cell. “Antisense”, when used in the context of a particular nucleotide sequence, refers to the complementary strand of the reference transcription product. “Antisense RNA” refers to an RNA transcript that is complementary to all or part of a target primary transcript or mRNA and that blocks the expression of a target gene. The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

A “protein” or “polypeptide” is a chain of amino acids arranged in a specific order determined by the coding sequence in a polynucleotide encoding the polypeptide. Each protein or polypeptide has a unique function.

It is to be understood that as used herein the term “transgenic” includes any cell, cell line, callus, tissue, plant part, or plant the genotype of which has been altered by the presence of a heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, the term “plant” includes reference to whole plants, plant organs (e.g., leaves, stems, roots, etc.), seeds, plant cells, and progeny of same. Parts of transgenic plants are to be understood within the scope of the invention to comprise, for example, plant cells, protoplasts, tissues, callus, embryos as well as flowers, stems, fruits, leaves, roots originating in transgenic plants or their progeny previously transformed with a DNA molecule of the invention and therefore consisting at least in part of transgenic cells, are also an object of the present invention.

As used herein, the term “plant cell” includes, without limitation, seeds suspension cultures, embryos, meristematic regions, callus tissue, leaves, roots, shoots, gametophytes, sporophytes, pollen, and microspores. The class of plants that can be used in the methods of the invention is generally as broad as the class of higher plants amenable to transformation techniques, including both monocotyledonous and dicotyledonous plants.

The successful transformation of Prunus with LAZY1 is a major step in manipulating tree shoot orientation and thus overall tree shape in Prunus, thus ensuring the development of improved varieties of Prunus that are more amenable to training or trellising.

### EXAMPLES

Having now generally described this invention, the same will be better understood by reference to certain specific examples, which are included herein only to further illustrate the invention and are not intended to limit the scope of the invention as defined by the claims.

### Example 1

### Arabidopsis Mutants

The Arabidopsis lazy1 mutant line previously described by Yoshihara et al. (2013. Plant J. 74:267-279) was obtained from the Arabidopsis Biological Resource Center (ABRC). The tac1 mutant line was previously described by Dardick et al., 2013. A double tac1;lazy1 homozygous mutant line was obtained via crossing. Double homozygous lines were confirmed via PCR using previously published primer sets (Ku et al. 2013, supra; Dardick et al. 2013, supra). All plants were maintained in were grown in 4 inch pots (one plant per pot) under fluorescent light (80 μmol m⁻²s⁻¹) in an environmental growth chamber set at 21° C. and 50% humidity unless otherwise specified.

An Arabidopsis knock-out mutant containing a T-DNA insertion within the LAZY1 gene was previously reported by Yoshihara et al. (2013, supra). We generated and characterized homozygous lines for this same mutant prior to that report in order to study potential functional relationships with TAC1. lazy1 Arabidopsis plants developed horizontally oriented lateral shoots as previously reported by Ku et al. (2011, supra) (FIG. 1). A tac1;lazy1 double mutant generated via crossing displayed a shoot phenotype identical to lazy1 alone, suggesting that the lazy1 phenotype is epistatic to tac1 (FIG. 1).

### Example 2

### Gravitropism Experiments

For gravitropism experiments, 4-5 week old plants having a single inflorescence shoot were adapted to dark conditions under green light illumination for 1 hr. Plants were then placed in a custom built rack which was rotated 90° to simultaneously stimulate shoot bending. Images were taken every minute for up to 24 hrs. The software Image J was used to quantify bending. Briefly, the curve of a circle is fit to the bent stem image and the area of the circle is calculated as an estimate of the stem arc. A minimum of 22 plants was used for each mutant and control line.

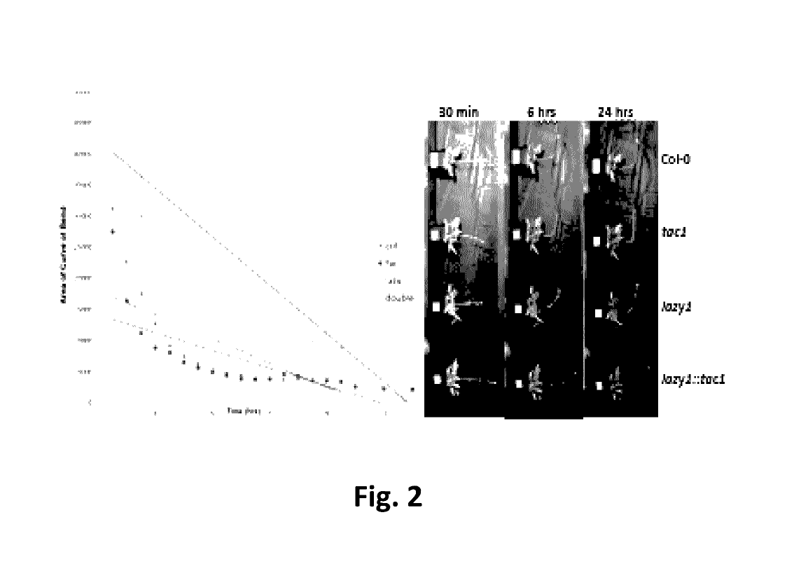

We performed comparative gravitropic bending experiments using tac1, lazy1, and tac1;lazy1 Arabidopsis mutants. The lazy1 mutant was previously reported to exhibit a complete loss of gravitropism (Yoshihara et al. 2013. Plant J. 74:267-279). Mature plants were adapted to conditions of green light for 1 hour prior to re-orientation (to avoid possible photostimulatory effects) and imaged under green light using time-lapse photography for up to 24 hours. Results showed that gravitropic bending was delayed in lazy1 plants rather than eliminated (FIG. 2). The tac1 plants displayed normal gravitropic responses; both the rate and degree of bending was statistically the same as wild type Col-O plants. Consistent with the epistasis observed in the lazy1-like phenotype, the double tac1;lazy1 mutant bent at the same rate as lazy1 plants (FIG. 2). Collectively these findings suggest that TAC1 does not contribute to gravitropism or contributes conditionally, possibly depending on environmental factors.

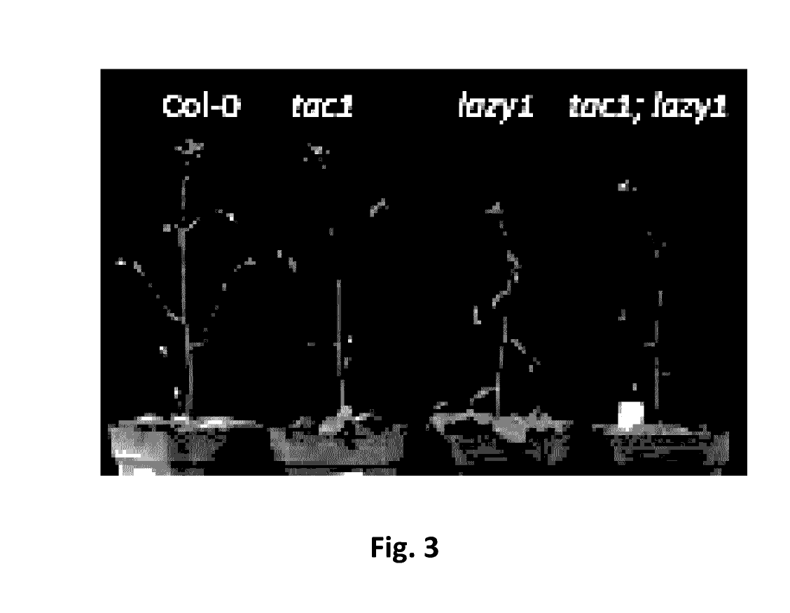

Arabidopsis mutants lazy1 and tac1;lazy1 display a weeping phenotype under high light. Phototropic responses of tac1 or lazy1 mutants have not been reported. However, we have found that lazy1 and tac1;lazy1 double mutants display a lateral shoot weeping phenotype under conditions of high light (˜170 μmol/m⁻²s⁻¹) (FIG. 3). These phenotypes suggest that LAZY1 phenotypes are light dependent.

### Example 3

### Plum Transformation

To generate LAZY1 silenced plum lines, a hairpin construct was created in pHellsgate 8.0 vector (Helliwell et al. 2002. Func. Plant Biol. 29(10):1217-1225). A 306 bp fragment (SEQ ID NO:4) corresponding to a portion of the peach LAZY1 gene (peach genome version 1.0 ID ppa007017) was amplified and cloned into the pENTR-D TOPO cloning vector (Invitrogen, Carlsbad, Calif.). The sequence in the resulting plasmid was next cloned into pHellsgate 8.0 plasmid using LR Clonase (Invitrogen, Carlsbad, Calif.). The pHELLSGATE 8.0 plasmid containing peach LAZY1 gene fragment was designed to silence LAZY1 and transformed into Agrobacterium tumefaciens strain GV3101. The gene construct was engineered into European plum (Prunus domestica L) following the protocol of Petri et al. (2012. Mol. Breeding 22:581-591). Cold (4° C.) stored seeds of ‘Bluebyrd’ plum were used for transformation. Briefly, the seeds were first cracked to remove stony seed coat, surface sterilized with 15% commercial bleach for 15 minutes, washed three times with sterile water, and the hypocotyl slices were excised from the zygotic embryos under a laminar flow hood using a stereomicroscope. After incubating for 20 minutes in an Agrobacterium suspension, the transformed hypocotyl sections were cultured for 3 days in co-cultivation medium. Finally the hypocotyl sections were plated in antibiotic (80 mg/i kanamycin) selection medium to produce transgenic shoots. The kanamycin resistant transgenic shoots were multiplied in plum shoot multiplication medium, rooted, acclimatized in the growth chamber and planted in 6-9″ pots in a temperature controlled greenhouse to evaluate growth and development.

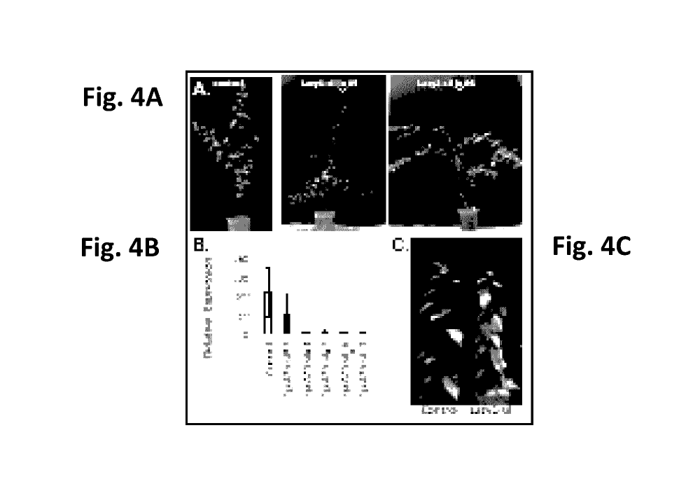

The resulting transgenic plum lines had horizontally oriented lateral branches and in some instances displayed a distinct weeping phenotype where both the apical and lateral branches grew rootward (FIG. 4). Leaves of LAZY1 sil plants also hung noticeably rootward (FIG. 4).

### Example 4

### LAZY1 Expression

Expression profiles of LAZY1 was assessed via qPCR analysis on an existing tissue-specific RNA collection (described in Dardick et al. 2013, supra). Total RNA was extracted from frozen tissue samples using E.Z.N.A SQ Total RNA Kit (Omega Bio-tek, Inc., USA), according to the manufacturer's instructions. Leaf tissue was used for extraction and for expression analysis in transgenic plums. Resulting RNA samples were then treated with DNase I to remove contaminating genomic DNA. qPCR reactions were carried out using SuperScript III Platinum SYBR Green One-Step qRT-PCR Kit with ROX (Invitrogen Corp., USA) and the reaction mix was produced according to the manufacturer's protocol. Gene-specific primers for the LAZY1 gene were previously reported (Dardick et al. 2013, supra). The qPCR was run using an ABI 7900DNA Sequence detector (Applied Biosystems) according to the following parameters: cDNA synthesis step at 50° C. for 5 min, followed by PCR reactions at 95° C. for 5 min and 40 cycles of 95° C. for 15 s, 60° C. for 30 s, and final 40° C. for 1 min. Standard errors of the means from three independent biological replicates were calculated.

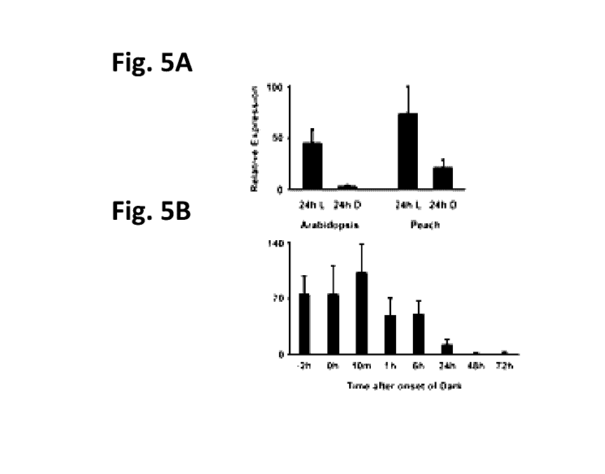

TAC1 but not LAZY1 gene expression responded to gravity and light stimuli Given the gravity and light dependent phenotypes associated with tac1 and lazy1, we tested the expression of these genes under different light conditions and after gravistimulation. TAC1 and LAZY1 are predominantly expressed in the upper portion of apical shoots in Arabidopsis and young peach trees (Yoshihara et al. 2013, supra; Dardick et al. 2013, supra). Twenty four hour dark treatment of Arabidopsis and peach plants led to significant suppression of TAC1 gene expression in the apical meristem (FIG. 5A). To further define the time interval for suppression a time course experiment was performed in Arabidopsis Col-0 plants. TAC1 expression decreased 6-24 hrs after the onset of dark and reached maximal repression by 48 hrs dark treatment (FIG. 5B). Unlike TAC1, LAZY1 expression remained unchanged under all conditions tested (data not shown).

### Example 5

### Protein Localization

Protein localization in onion epidermal cells was done using a modified version of the protocol described by Xu et al. (2014. PLoS One 9(1):e83556). Agrobacteria GV3101 containing PpLAZY in pEarleyGate103 was grown in 50 mL LB media containing 20 μM acetosyringone (AS) to an A₆₀₀ of ˜1.5. Next, the bacteria was pelleted, washed 2-3 times with infiltration media (41.65 mM Dextrose, 100 mM CaCl2, 100 mM MES-KOH, 0.011 μM BAP, 200 μM AS, 0.01% Silwet L-77, 0.05 mM MgCl₂) before being re-suspended to a final A₆₀₀ of ˜0.1 in infiltration media. After incubating at room-temperature for several hours, the cell suspension was infiltrated into segments of onion layers still attached to the bulb using an insulin syringe. The onion was wrapped with a rubber band to hold the segments together and then incubated in the dark at 28-30 C for 2 to 3 days. Epidermal layers were peeled off onion sections, placed on a slide with water, and topped with a coverslip before imaging with an Axio Zoom microscope (Zeiss, Okerkochen, Germany).

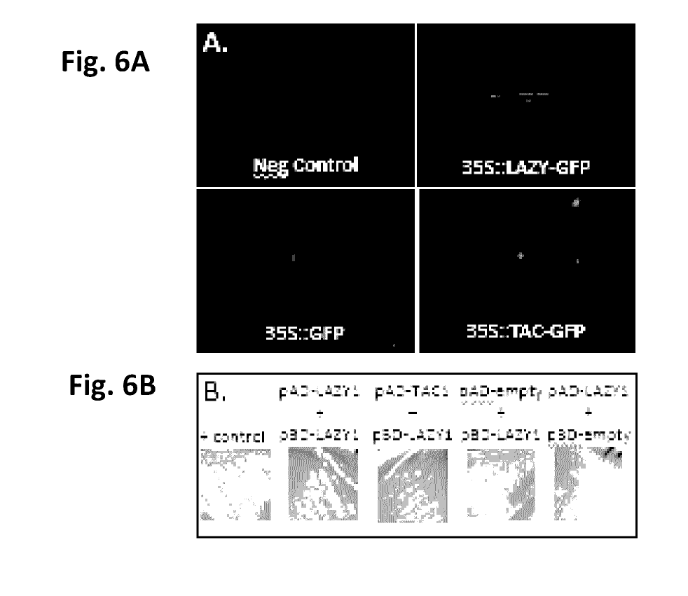

TAC1 and LAZY1 proteins show similar subcellular localization patterns. GFP-fusions of Arabidopsis, rice, and corn LAZY1 proteins were previously shown to localize to both the plasma membrane and the nucleus using transient assays (Li et al. 2007, supra; Dong et al. 2013, supra). Yoshihara et al. (2013, supra) found that mutation of the putative nuclear localization signal did not alter its function. A more recent report found microtubule localization of LAZY1 (Sasaki and Yamamoto. 2015. Plant Biotech. e-print). To our knowledge, the localization of TAC1 has not been reported. We performed localization studies in onion epidermal cells to compare TAC1-GFP and LAZY1-GFP and found that TAC1-GFP showed the same pattern as LAZY1-GFP, localizing to the plasma membrane and nucleus (FIG. 6A).

### Example 6

### Yeast Two-Hybrid

To generate the yeast-two-hybrid Binding Domain (BD) vectors with the pXDGAT backbone (Ding et al. 2004. Anal. Biochem. 331(1):195-197), full length peach LAZY1 CDS sequence was amplified by RT-PCR from peach RNA using the following primers: FL-LAZY-for-YTH-F: 5′ CACCATGAAGTTACTAGGTTGGATGCATCG (SEQ ID NO:5) and FL-LAZY1-for-YTH-R: 5′ CTTCATTTCACCTTTCACAGCTCC (SEQ ID NO:6). The resulting amplicon was TA-cloned into the pENTR/D-TOPO vector (Invitrogen, Carlsbad, Calif.) and then cloned into pXDGAT via LR-reaction to produce pLAZY-BD. Yeast-two-hybrid Activation domain (AD) constructs were made using the pGAD424 AD vector (Clontech, Mountain View, Calif.). The peach LAZY1 CDS sequences was amplified from pENTR/D vectors (described above) using the following primers: PpLAZY-CDS-EcoRI-F: 5′ GAATTCATGAAGTTACTAGGTTGGATGCAT (SEQ ID NO:7) and PpLAZY-CDS-BamHI-R: 5′ GGATCCTCACAGCTCCAAGACTAAGTAGT CT (SEQ ID NO:8). The resulting amplicon was TA-cloned into the pCR8/GW/TOPO® TA vector and digested with BamHI and EcoRI before ligation into the corresponding restriction sites in the pGAD424-GAL4 plasmid. To generate the GFP-tagged overexpression vectors, the LAZY1-CDS was amplified from the PpLAZY-AD (pGAD424) vector using primers PpLAZY-pEG-F: 5′ ATGAAGTTACTAGGTTGGATG CAT (SEQ ID NO:9) and PpLAZY-pEG-R-NoStp: 5′ CAGCTCCAAGACTAAGTAGTC TGC (SEQ ID NO:10). The resulting amplicon was first TA-cloned into the PCR8/GW/TOPO vector (Invitrogen, Carlsbad, Calif.) and then moved into pEARLEYGate103 via LR-Reaction (Earley et al. 2006. Plant J. 45(4):616-629).

The yeast-two-hybrid pXDGAT and pGAD424 vectors were transformed one at a time into the yeast strain AH109 according to the Clontech Yeast Protocols Handbook PT3024-1 (Mountain View, Calif.). Selection for yeast containing both plasmids and for protein-protein interaction was done on −Trp/−Leu and −Trp/−Leu/−His plates, respectively, both containing 25 μg/mL tetracycline.

LAZY1 homodimerizes but does not interact with TAC1 in yeast. To test if peach TAC1 and LAZY1 interact with themselves or each other, a yeast two-hybrid experiment was performed. Yeast transformed with both the LAZY1-AD and LAZY1-BD plasmids grew on selective media, while either plasmid transformed in combination with empty vector controls did not confer growth. In contrast, LAZY1-BD did not show any significant interaction with TAC1 (FIG. 6B)

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

We claim:

1. A cDNA molecule comprising 306 consecutive nucleotides (SEQ ID NO:4) encoding a fragment of the LAZY1 polypeptide (SEQ ID NO:2) of Prunus.

2. An isolated or recombinant 306 consecutive base pair polynucleotide fragment (SEQ ID NO:4) of the LAZY1 gene (SEQ ID NO:1).

3. A RNAi construct comprising the isolated 306 consecutive base pair polynucleotide fragment of claim 2.

4. A vector comprising the LAZY1 RNAi construct of claim 3 wherein said construct comprises the nucleotide sequence (SEQ ID NO:4) of about 306 consecutive base pairs of a fragment of the LAZY1 gene of Prunus.

5. A host cell comprising the LAZY1 RNAi vector construct of claim 4.

6. The host cell of claim 5, wherein said host cell is a Prunus cell.

7. The host cell of claim 6, wherein said host cell is a cell from any one of Prunus persica, Prunus domestica, Prunus avium, Prunus salicina and Prunus armeniaca.

8. A method of producing a Prunus plant having the characteristics of weeping phenotype or horizontally oriented branches comprising: constructing a recombinant vector comprising a construct comprising the 306 base pair consecutive nucleotide fragment (SEQ ID NO:4) of the LAZY1 gene of Prunus, transforming Prunus plant cells with the recombinant vector and expressing in the plant said construct encoding the LAZY1 gene sequence comprising a 306 bp consecutive sense nucleotide fragment of the LAZY1 gene of Prunus and the antisense-complement thereof, wherein the expressing induces RNA interference (RNAi) in the plant resulting in plants having the characteristics of a weeping phenotype while maintaining quality and yield characteristics of flower or fruit development of a non-transformed Prunus plant.

9. The method of claim 8 wherein said method results in plants having the characteristics of a weeping phenotype and thereby exhibiting improvement in quality and yield characteristics of flower or fruit development compared to a wild-type non-transformed Prunus plant.

10. The method of claim 8 wherein said method results in plants having the characteristics of a weeping phenotype and thereby allowing for increased planting density and increased soil utilization compared to a wild-type non-transformed Prunus plant.

11. A transgenic Prunus plant produced by the method of claim 8 or the progeny thereof, said plant exhibiting changed plant architecture with horizontally oriented branches compared to a wild-type non-transformed Prunus plant.

12. A transgenic Prunus cell comprising the RNAi construct of the invention, wherein the transgenic plant regenerated from said cell exhibits suppression of the LAZY1 gene, resulting in a plant demonstrating changed plant architecture with horizontally oriented branches and leaves having a rootward orientation, relative to the wild-type Prunus plant.

13. A transgenic seed of the transgenic plant of claim 11, comprising the LAZY1 RNAi construct of the invention.

14. Plants, plant cells, and plant parts, and plant seeds from any one of Prunus persica, P. domestica, P. avium, P. salicina and P. armeniaca which have been transformed by the LAZY1 RNAi construct of claim 3.


SEQUENCE LISTING (10 sequences)

SEQ ID NO: 1 - DNA, 1170 bp, Prunus domestica
atgccaatgttgcagttactaggttggatgcatcgtaagtttcggcagaatagcaacgagccatttaaagtttttgtcattgggcagccatctctcgatgatcaacaatgctatcctaagccaaactgtggcacaaagccctttaaacaaacccagagagaccagcaccttcggaagtctttcaacggtctagaggcagccagggcagaagaagaatactatgaagatgaatcatctgctgcagcatctgagctcttccatggcttccttgcaattggtacccttggctcagagcaagtcatcacggaaccatcaactccaacacttgccatctctgtggagaacataactgaaaaagagactgaggtcacagagaatgaattgaagctcatcaatgatgaattggagaaagtcctggcagctgattcagctaaagatgagatttgcaatgattcatctggaagaaacagccatgttagcaatggaagaagtagccatggtagcaccatcacactaagtggcaagacactggaaggctcagagagcaatgggattaatggaaccacagtgtgcccactccagggatatctttttgggtcagcatatgaattgtcagaaacaacaacagtggcaaagaaggaacacaggacatctcttggcgagctgtttcagaggactaaattggcagaggagatttctggaccgaaatcggccaaggaggagaagcgagcagagaaggaagctgaaaagtccgccatgcacttgatgaaaaagaagctcaagaaaaaaatgctttatgcttcttctcgcagctctggtggacctgcagatccttcctcagcggaaacaaaactgaataagatccttcacatgttccacagaaaagttcaccctgaaacctcatctgctgagcaaaaaactggtaagtaccataagaacgaaaacaagaagaaaacaagcaatgatggggcttacaacagtggagatcaggtgcttccagatgaagacatcatgctatatcctgaacgaggcttctccttgaagcagagcatgcggcgctacaagagtcaatcaaacccaccgcaattcgcgcttagcagcattgattcaaatgagaacagggagcactggatcaaaacagatgcagactacttagtcttggagctgtga

SEQ ID NO: 2 - protein, 389 aa, Prunus domestica
MPMLQLLGWMHRKFRQNSNEPFKVFVIGQPSLDDQQCYPKPNCGTKPFKQTQRDQHLRKSFNGLEAARAEEEYYEDESSAAASELFHGFLAIGTLGSEQVITEPSTPTLAISVENITEKETEVTENELKLINDELEKVLAADSAKDEICNDSSGRNSHVSNGRSSHGSTITLSGKTLEGSESNGINGTTVCPLQGYLFGSAYELSETTTVAKKEHRTSLGELFQRTKLAEEISGPKSAKEEKRAEKEAEKSAMHLMKKKLKKKMLYASSRSSGGPADPSSAETKLNKILHMFHRKVHPETSSAEQKTGKYHKNENKKKTSNDGAYNSGDQVLPDEDIMLYPERGFSLKQSMRRYKSQSNPPQFALSSIDSNENREHWIKTDADYLVLEL

SEQ ID NO: 3 - DNA, 4593 bp, Prunus domestica
gcatacagaagttgagattacaaaagcaatacaaaataaacctatcccacaaaccccattcttctgattactcattgtcaagtgctgttctatccagctttataagataacctacgtattaaacagatgaatcagagcaccgttttgggaattattatcataaaaacaagaatcagccactatcatcaacagttacccgatccaaggccatgccccctgctattcaaaaaatcttatcaaccagtaaagtggaatctcaacataccatgtagatggaaataaaacataaggagactgccaatctaacagtagcgcaattatttgtgattgatatccactctacccctatttataaacattttcaatcaatctgaaaatatatcttggaatttttcaagggttttgtttgtgccacagaaaaaattaacataatcattgaaagagaacattaacccttcttaagctttttctctagcttgctggtacctatgcgtagagattagacaaagatgcgtctggacattaagagagcacaatggctagatgcctctaggctctagagatgatcttatccgttatccccacttgattttgttcatgttgccacagaccatagaatacagaggcaatggaaaagaaacagatcaagtttgcatccatcattttcacagaaacaaattcttctacttctacaaacataaaaagattcttcaatgaaattattgtcccttgattaatcatttatcagttattactgaaattgcacaaaaatgcgttttctttggttcctatcaaggacgcttcagttagactgttacctttgtctctaagccctcaaaccctttagtttttatcagctcctccttattcatatatgtagatgctcgcggaacttttccaggacaaaacggccccgttccacatgcacatttctgactctgagagtgaacacgtttctaatgaaaacgatatttgcaagtacttgaacatgaatggttaataatgagggaggacaacttgataacatgtggcaaccttgccatctcatgagtcatgtcaaaaggtttcaggaatattgacgagataaaagactttatgcaggaaccttttttttcttgttctttttctagtaaatcttatgccagaatggtggtgaaaaatgaatgtcattgcctgcaaaagcactgaaaacaccttgaactagtttataagaccggctagaactatcaaacagaaaacatgacagcagtggaagatgtggagacatacctgcattaaatcaccaatcccatcaaagcttataagctgatatcctcgaaacatggtagaatatgggatttatttggagttcaatggtaaattgattgcaaacagcaaaagaaaaaaagtttaaccataggaagatacttgttaaaagcaatcccagtttatcatagtttatcagctaagctctttcggtaagaactccacctgtggacctaccaacagtagagagtccgacgttttgtgtccccagagaaagaagatcaacttaattgtcgaattatatgtttggctccattgaggtccgacaaaatataccttgcagtccctactgttcaatctagggtcttggcacaagttcctagtaaaataacaaagtagtagaaattattagacgaaacttactttgttatcctctgcccaaagcatgtgcatgatgaacatgtcagcgtggaaagcccaaatgtaacatggtactcctgatatgggcatgtttttcaaaacaggacgtgcacagttgcatcagaaaacaccacatccagaacacagaagacatagcagatatacgtgtatgtgtgtgtgtagatatacatgtttttcaaaacatgtgcacaacacatcaacactgtgtatactgaaagacgtgtaattatgcaccaaatgattataaaatataaaccctataacaatcaatctcacacaataaacatatctgcatgactgggttggtggtccgggacccacttgccatttgtccacttgatttgatcctgacaaagaatagccaggccaagtttgtaactatctgtgtgtactcctccgctcacctgcttttcagagcctggaggctttcaggtggcatttgacatcttcttcataaataagcattaagactacctatggaaagaagctcctctgtcaacctctcctaggaaaagaattggatcaaagctctcaagtgttctaaacaagatcataagatgaaggtaagaaaagcctattctcttattatcctttagataagctaaaactgcttcaactaatctataatttgatcttgcatgccaatgttgcagttactaggttggatgcatcgtaagtttcggcagaatagcaacgagccatttaaagtttttgtcattggtaagtcgtgtggtgccttatgcctttctcctatgcattcctgtataactgcagtttaccaaaagaacggcagaaaacatcactttgggttctaatgatgcacagcaagtttgtgaatcaaccagataaatcatttcattcctgaaagacggtacaggatgcaactaagactttgtctgcctaataaaagaaattagtttcgaccaaaaaaaaataaaagaaattagtctgaacaaggatttggtgaaatacagtagtttccccattgcatgatttgtaatactagaacagaacagccccaaaataaatcacatgaagtacatatgcgtaacagcttgggaaccaaaagactaatatgtacttagaaatcataagatcattattagagacagagaggtttgtaccgatatgcagtgggagaatctcttttcttgctcaatctgagtctcatccaagtttacattttcttctttgtgtcagagaattcttgtgcttgtctttcagggcagccatctctcgatgatcaacaatgctatcctaagccaaactgtggcacaaagccctttaaacaaacccagagagaccagcaccttcggaagtctttcaacggtctagaggcagccagggcagaagaagaatactatgaagatgaatcatctgctgcagcatctgagctcttccatggcttccttgcaattggtacccttggctcagagcaagtcatcacggaaccatcaactccaacacttgccatctctgtggagaacataactgaaaaagagactgaggtcacagagaatgaattgaagctcatcaatgatgaattggagaaagtcctggcagctgattcagctaaagatgagatttgcaatgattcatctggaagaaacagccatgttagcaatggaagaagtagccatggtagcaccatcacactaagtggcaagacactggaaggctcagagagcaatgggattaatggaaccacagtgtgcccactccagggatatctttttgggtcagcatatgaattgtcagaaacaacaacagtggcaaagaaggaacacaggacatctcttggcgagctgtttcagaggactaaattggcagaggagatttctggaccgaaatcggccaaggaggagaagcgagcagagaaggaagctgaaaagtccgccatgcacttgatgaaaaagaagctcaagaaaaaaatgctttatgcttcttctcgcagctctggtggacctgcagatccttcctcagcggaaacaaaactgaataaggtacctctgatcaaacattgtgcgtgcatttcagacttttcacatcaaaagagtaatgtgaactgaattagaccatatagtcctggctcatggctatggtgtgtgtgtggcttgcagatccttcacatgttccacagaaaagttcaccctgaaacctcatctgctgagcaaaaaactggtaagtaccataagaacgaaaacaagaagaaaacaagcaatgatggggcttacaacagtggagatcaggtgcttccagatgaagacatcatgctatatcctgaacgaggcttctccttgaagcagagcatgcggcgctacaagagtcaatcaaacccaccgcaattcgcgcttagcagcattgattcaaatgagaacagggagcactggatcaaaacagatgcagactgtaagtacactctgcactaagctcttaatgtagtatttggtgcctgaatgcctttacttaacaatgaacactaaaatgaatcaagtattacattaactgtgaatcaaagttttccctattctttagaccatcctaattcttgtgttgtgttgtgtttgctgtagacttagtcttggagctgtgaaaggtgaaatgaagaagatggctttccagttgggttttcaaattaattgtgaagcttgatcagtaagaaagaagtgattatagttgctgtttgtaagagtgagattttactttgttggtgttacttagtagtgttggcatttgccaataatgatcaaattaaagagcatttgaagaagagataaataaaaagaggcgctcagtcctcttataaatctatctgattggtgaatgtaaaatatgtaagatcgtgtactttggctggtcatcgtatttcaacaattatcagaaattttgaaatatatttttatacgt

SEQ ID NO: 4 - DNA, 306 bp, Prunus domestica
aagccaaactgtggcacaaagccctttaaacaaacccagagagaccagcaccttcggaagtctttcaacggtctagaggcagccagggcagaagaagaatactatgaagatgaatcatctgctgcagcatctgagctcttccatggcttccttgcaattggtacccttggctcagagcaagtcatcacggaaccatcaactccaacacttgccatctctgtggagaacataactgaaaaagagactgaggtcacagagaatgaattgaagctcatcaatgatgaattggagaaagtcctggcagct

SEQ ID NO: 5 - DNA, 30 bp, Artificial Sequence
  Chemically Synthesized
caccatgaagttactaggttggatgcatcg

SEQ ID NO: 6 - DNA, 24 bp, Artificial Sequence
  Chemically Synthesized
cttcatttcacctttcacagctcc

SEQ ID NO: 7 - DNA, 30 bp, Artificial Sequence
  Chemically Synthesized
gaattcatgaagttactaggttggatgcat

SEQ ID NO: 8 - DNA, 31 bp, Artificial Sequence
  Chemically Synthesized
ggatcctcacagctccaagactaagtagtct

SEQ ID NO: 9 - DNA, 24 bp, Artificial Sequence
  Chemically Synthesized
atgaagttactaggttggatgcat

SEQ ID NO: 10 - DNA, 24 bp, Artificial Sequence
  Chemically Synthesized
cagctccaagactaagtagtctgc